(19) United States
(12) Patent Application Publication
(10) Pub. No.: US 2004/0162491 A1
(43) Pub. Date: Aug. 19, 2004

(54) Method and device to localize light-emitting regions

(76) Inventors: Marcus Pfister, Erlangen (DE); Bernhard Scholz, Heroldsbach (DE)

Correspondence Address: SCHIFF HARDIN, LLP, PATENT DEPARTMENT, 6600 SEARS TOWER, CHICAGO, IL, 60606-6473, US

(21) Appl. No.: 10/722,030

(22) Filed: Nov. 25, 2003

(30) Foreign Application Priority Data
Nov. 25, 2002 (DE) 102 550 13.1

(51) Int. Cl. A61B 5/00

(52) U.S. Cl. 600/476

(57) ABSTRACT

In a method and a device to localize regions in a biological tissue section, at least during the examination the tissue section exhibits one fluorescence property differing from the tissue section, due to which, given an exposure with light of a first wavelength, light of another wavelength is emitted.

A sequence of fluorescence-exciting light signals at different locations on the tissue-section is applied. Fluorescence light is measured at a number of measurement locations on a surface of the tissue section, which appears there due to the light signals. Frequency-independent signal portions in the response signals are determined and are further processed into input values of a localization step. The tissue section is modeled and a set of guide fields is determined. The guide fields are transformed that in a localization step the frequency-independent signal portions are compared with the transformed guide fields, and the location of the transformed guide fields that best reproduce the frequency-independent signal portions is output as the location of the region to be localized.

### BACKGROUND OF THE INVENTION

1. Field of the Invention

The present invention concerns a method as well as a device to implement the method to localize regions, in particular focal lesions in a biological tissue section that, at least during the examination, exhibits a fluorescence property distinct from the tissue section, due to which, given an exposure with light of a first wavelength, light of another wavelength is emitted.

2. Description of the Prior Art

In Umar Mahmood et al., “Near Infrared Optical Imaging of Protease Activity for Tumor Detection”, Radiology 213:3, 866-870 (1999), it is specified that fluorescing metabolic markers either accumulate exclusively in specific regions, for example tumors, inflammations or other specific metastases, or are distributed throughout the body, but are activated only in specific regions, for example by tumor-specific enzyme activities and by additional exposure by means of light.

This principle of optical fluorescence imaging is explained using FIG. 1, in which a tumor is visible given the exposure with NIR light (light in the near-infrared range) after a marker whose fluorescence properties are activated by specific enzymes was administered to a mouse.

The recognition of a tumor or another marked region then ensues by exposure of the region with light in the special excitation wavelengths of the fluorescent dye, and detection of the emitted light in the corresponding emission wavelength of the fluorophore. Given authorization for use on humans, these markers can be used in early cancer detection, for example.

By exciting the dye with at least one temporally varied excitation light signal, for example by temporal variation of irradiation location and/or light wavelength and/or intensity modulation of the excitation light, data can be acquired (for example by means of CCD or photomultiplier, photon flux on the surface of the tissue section, such as, for example, the female breast) at different measurement points at different variations. In this manner, variation-dependent and location-dependent—meaning spatially two-dimensional—measurement data are acquired. In the case of M measurement data of N variations, these are M×N data.

From these data, spatially delimited lesions such as, for example, focal fluorescing marked tumors with different fluorescence properties than the surrounding tissue can be located three-dimensionally.

The method is provided in cancer (screening) examinations of the breast, lymph nodes, thyroid, prostate, and has intraoperative applications, and in general is applicable for all organs near the surface that lie in the range of the penetration of light and that develop carcinoma (or other diseases) for which (now or at a future point in time) corresponding fluorescent markers exist.

Various approaches are known for fluorescence reconstruction or localization.

Britton Chance proposed a method to localize fluorescing absorbers in homogenous medium known as phased arrays. This method localizes fluorescing inhomogeneities (spots) only in absolutely homogenous media, meaning media with homogenous light absorption properties and scatter properties (as they rarely occur in the application), and offers no information at all about the depth at which the spot is located.

Otherwise, various methods for fluorescence reconstruction have been proposed. In the reconstruction, the complete fluorescence activity in the entire (mostly discretized) medium is determined (similar to methods in nuclear medicine), while in the localization exclusively the regions emphasized from the background are sought. Reconstruction methods thus are based on the (often iterative) solution of large equation systems and are thus, in contrast to the localization operating in real-time that is proposed here, very time-consuming. The reconstruction methods further predominantly assume that the medium (similar to computer tomography) to be examined is enclosed by a ring of light sources and detectors.

Some of the known methods are tomography with frequency-modulated light (U.S. Pat. No. 6,304,771 and U.S. Pat. No. 5,865,754), which requires a reconstruction time of 5 min. on a 1 GHz Pentium computer or 45 min. on a SUN Sparc 2 workstation, and tomography with light (PCT Application WO 02/41760) that likewise requires a reconstruction time of 5 min. on a 1 GHz Pentium computer.

All of these known methods are characterized by a high calculation effort and relatively small reconstruction volumes; a calculation in real-time is not possible.

### SUMMARY OF THE INVENTION

An object of the present invention is to enhance the localization precision fluorescing marked tumors in a localization method of the above-described type as well as a device to implement the method, as well as to enable an evaluation in deep tissue slices, and to drastically reduce the calculation expenditure and, as a result, the calculation time.

With the inventive method, the problem of the localization of fluorescing subjects in optically blurred media can be quickly solved. Furthermore, the precision is increased via the variation of the excitation location.

In the inventive method light emission of tissue sections in which fluorescence markers are accumulated is excited by irradiation by laser light of suitable wavelength. Fluorescence light can then be measured at the proximate skin surface. In order to determine locations and optical parameters of marked tissue sections, a sequence of fluorescence excitations of the surface, for example of various locations with different modulation frequencies (including zero frequency), is radiated into the tissue, and then the fluorescent light is measured with one or more arrangements of suitable light sensors distributed on the surface, in order to thus acquire two-dimensional measurement value distributions which are dependent on the type of the excitation.

In accordance with the invention, frequency-independent signal portions are determined in the response signals, obtained by measuring the fluorescence light, and these frequency-independent signal portions are further-processed into input values for localization. The tissue section is modeled and a set of guide fields is determined from the model. The guide field are transformed, and the transformed guide fields are compared with the input values processed from the frequency-independent signal portions. A location of the transformed guide fields that best reproduces the frequency-independent signal portions is emitted, as an output, as a location of the region to be localized.

It has proven to be advantageous when, to generate the various fluorescence properties, the regions are marked with fluorescing markers (fluorophores).

The spatial resolution is enhanced when the fluorescence-exciting light signals are generated with various modulation frequencies and are irradiated into the tissue section.

It is advisable to first normalize and then transform the guide fields, whereby the guide fields can be transformed into orthogonal guide fields. Furthermore, the orthogonal guide fields can be determined from the guide fields by a singular-value decomposition.

The optical parameters can be determined in accordance with the invention by reference measurements by means of estimating methods, given non-fluorescence-exciting wavelengths.

A device for implementing the above-described method has at least one arrangement of light sensors distributed on the surface of the tissue section to measure the fluorescence light emitted by the fluorescing marked region, and laser diodes are provided to generate the light for exciting the marked region, so that a two-dimensional measurement value distribution is obtained as a result of the excitation. The output signals from the sensor arrangement are supplied to a processor for implementing the above-described method to localize the region or regions.

A measurement system can, for example, include 8×8 regular light sensors arranged on a planar measurement surface. However, it can be advantageous to measure with a number of such planar systems at the same time. Thus, for example, two arrangements of light sensors can be provided that can be applied on both sides of the tissue section to be examined. Given measurements of the female breast, two measurement surfaces can be applied to opposite sides of the mamma. An advantageous embodiment is the integrated arrangement of the measurement surfaces in pressing plates of an x-ray mammography device.

In general, any curved or curvable or flexible measurement surface can be used with any arrangement of light sensors.

### DESCRIPTION OF THE DRAWINGS

FIG. 1 is exposure to explain the principle of optical fluorescence imaging.

FIG. 2 is an overview of the basic components of a device to localize and classify a focal lesion in a tissue section in accordance with the invention.

FIG. 3 shows the substantial method steps to localize a focal lesion in accordance with the invention.

FIG. 4 is a schematic illustration of an inventive applicator with 8×8 sensors as well as 8 light sources arranged near the measurement surface to generate the excitation light.

FIG. 5 is a schematic illustration of a double system in accordance with the invention with two applicators positioned opposite one another.

FIG. 6 is a two-dimensional measurement value distribution of a data configuration designated as the configuration 1, for the first four excitation locations.

FIG. 7 shows a singular-value decomposition of the configuration 1.

FIG. 8 is a basis map of the configuration 1.

FIG. 9 shows localization functions of two fluorochrome-marked lesions.

FIG. 10 illustrates the localization of lesions of different depths with a planar measurement system.

FIG. 11 illustrates the localization of lesions of different depths with two planar measurement systems placed opposite to one another.

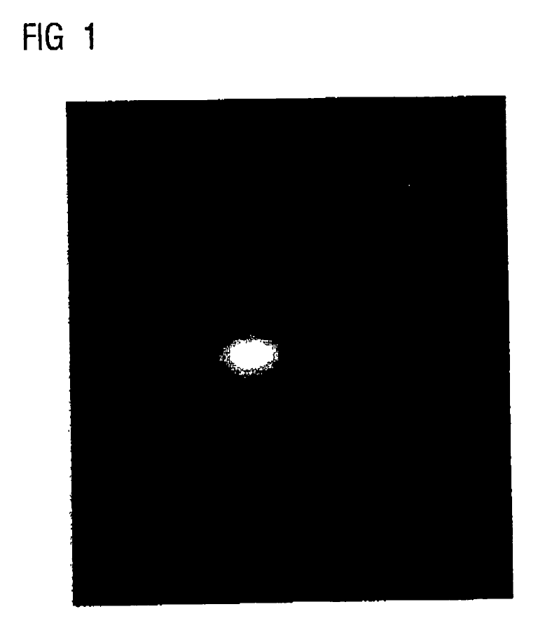

### DESCRIPTION OF THE PREFERRED EMBODIMENTS

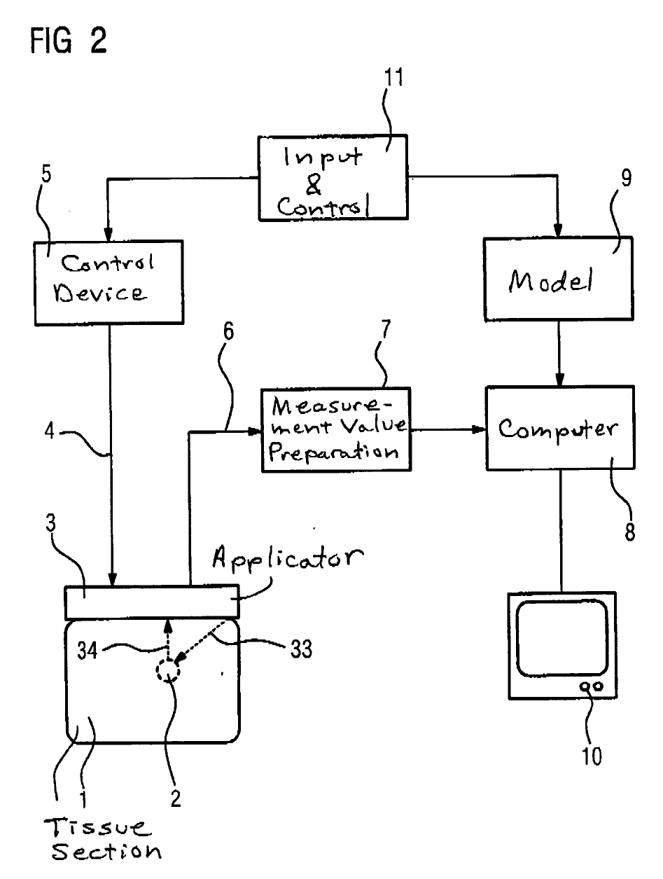

The overview representation in FIG. 2 shows a measurement and evaluation arrangement with which a delimited spatial area 2 arranged in a biological tissue section 1 can be localized and identified. It is assumed that the spatial area 2 possesses a fluorescent property different from the remaining tissue section 1. These assumptions are fulfilled sufficiently well when the biological tissue section 1 is a female breast and the delimited spatial area 2 is a tumor to which, for example, a fluorescing metabolic marker was supplied whose fluorescent properties are activated by specific enzymes.

The measurement arrangement includes an applicator 3 with a number of spatially distributed photo sensors, as well as additional laser diodes arranged in a line.

The recognition of a tumor or another marked region ensues by exposure of the region with light of the laser diodes in the specific excitation wavelength of the fluorescent dye, and detection of the emitted light by the photo sensors in the corresponding wavelength of the fluorophore.

The photo sensors and laser diodes of the applicator 3 are connected via electrical connection lines 4 with an electrical control device 4, and with a measurement value processor 7 via electrical connection lines 6.

With the control device 5, pulses of NIR light are supplied to the biological tissue section 1 via a number (K) laser diodes, whereby 1≦K≦M, in order to excite to fluorescence an existing marked tumor.

To localize and identify spatially delimited areas 2, the light emitted from the areas 2 is measured by photo sensors on the surface of the tissue section 1 at M locations and supplied to the processor 7.

The measurement value processor 7 includes, for example, measurement amplifiers, filters and A/D converters. The measurement value processor 7 is connected with one or more data inputs of an electronic computer 8. In addition to the measurement values, a model 9 of the tissue section 1 is available to the computer, with which the above-cited fluorescing areas 2 are localized and identified, as is specified further below. The result, for example in the form of a graphical representation of the anatomy of the tissue section wherein the location of the light sources (and thus of the spatial areas 2) is marked, ensues via a monitor 10. Since the calculation, among other things, is determined by the model 9 and the location of the exposure, a supervisory input and control 11 is provided with which the number and the location of the photo sensors are determined, as well as the number and location of the laser diodes, the value of the frequency, and the model.

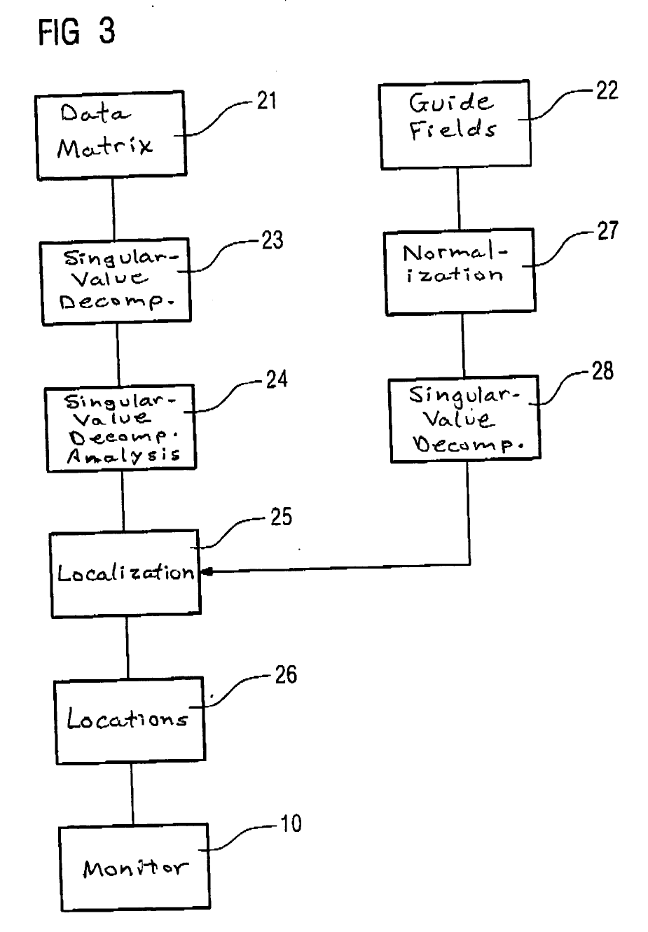

The localization method is explained using FIG. 3 as an example. Explained first are its input dimensions, meaning the measurement data and model data, and then the calculation steps of the method.

The input dimensions for the localization method are, per measurement surface.

a) An M×N data matrix D with measurement values (reference number 21) which are dependent on the M sensor locations \overline{r}_{S,m}, (m=1, . . . , M) and the N excitation parameters (N₁ excitation locations \overline{r}_{A,n}_{₁}, (n₁=1, . . . , N₁) and/or N₂ excitation modulation frequencies fₙ_{₃}, (n₂=1, . . . , N₂) whereby N=N₁+N₂), and which can result from the actual measurement data by post-processing.

The M-dimensional column vectors of the data matrix can be reformatted corresponding to the arrangement of the sensors on the measurement surface. A graphical representation of the reformed column vector provides a visualization of the measurement value distribution over the considered measurement surface for a given excitation type. In the case of the above cited 8×8 sensor distribution, the 64-dimensional column vector is reformed into an 8×8 matrix.

b) A set of K guide fields or lead fields Lₖ(\overline{r}ₘ,\overline{n}ₘ,\overline{r}₁,μₐ,μ_{z}),(k=1, . . . , K), for example multipole lead fields which are characterized with the reference number 22 in FIG. 3, and which for their part are dependent

the model of the optical medium of the examination area 1,

the measurement system, for example location \overline{r}ₘ and/or normal vector \overline{n}ₘ of the m-^{th} sensor,

the location \overline{r}_{f} of the f-^{th} excitable fluorochrome,

the type of the measurement (frequency modulation yes/no) and

optical parameters such as the absorption and scatter coefficients μₐ,μ_{z} of the medium surrounding the lesion(s).

The photons impacting on a sensor are transduced (converted) into electrical signals and then supplied to the further evaluation. In the case of frequency-modulated excitation, light intensity and phase shifts with regard to the input wave are measured. Both real measurement values can be combined into a complex measurement value. The data matrix is then—in the mathematical sense—complex. In the following, in the general case a complex data matrix is assumed.

It may be necessary to supply post-processed measurement data to the localization algorithm. For example, edge artifacts are eliminated via truncation of edge data. They could simulate a nonexistent dependency on the modulation frequency location or the excitation frequency.

The data matrix can result from a linear combination of at least two data sets. For example, the difference of a data set with fluorescence signals and a spatially adjacent data set without fluorescence signal can be considered. It is to be expected that possible contributions of background excitations are reduced in the difference data, if not completely eliminated.

Guide fields, known as lead fields, are known quantities from bioelectric magnetism. They describe the measurement value distribution of a standard signal source that can be acquired with a given measurement system.

Lead fields, which specify the light intensity that can be acquired with a measurement system or a number of measurement systems based on optically excited focal lesions marked with fluorochromes, are suitable as input quantities for the method to localize such focal lesions.

For example, in the exemplary embodiment only a lead field is used. It describes the light intensity of a punctiform light source measurable with a given measurement system. Corresponding to the expanded electrically-polarized lesion areas addressed in B. Scholz, “Towards Virtual Electrical Breast Biopsy: Space-Frequency MUSIC for Trans-Admittance Data”, IEEE Trans. Med. Imag., Vol. 21, No. 6, pp. 588-595, spatially expanded fluorescence sources can likewise be acquired by multipole lead fields. In the following, it is assumed that exemplary lead fields, meaning a set of a number of lead fields, are available.

For the further steps, it is helpful to combine the values of the k-^{th} lead field Lₖ(k=1, . . . , K) at the M measurement locations into an M-dimensional vector in data space (symbolized by the underline under L).

Lₖ(\overline{r})=(Lₖ(\overline{r},\overline{r}₁), . . . , Lₖ(\overline{r},\overline{r}_{M}))^{T} (1)

with k=1, . . . , K

wherein \overline{r} is the focal point of the lesion. For clarity, in equation (1) the dependency on the optical parameters of the medium surrounding the lesion(s) is not specified.

The optical parameters that, as noted above, enter into the lead fields, can be determined by reference measurements by means of estimation methods, given non-fluorescence-exciting wavelengths.

The signal processing of the method involves per measurement surface

1. the singular-value decomposition of the data matrix D (reference number 23 in FIG. 3),

2. the analysis of the singular-value decomposition (reference number 24 in FIG. 3), and

3. the actual localization method (reference number 25 in FIG. 3).

The singular-value decomposition 28 of a matrix is a known mathematical method from G. Golub, Ch. Van Loan, Matrix Computations, 3rd edition, J. Hopkins University Press, 1996, Page 70 et seq. For the above data matrix, the singular-value decomposition is

D=U S V^{H} (2)

wherein

U a unitary M×M matrix dependent only on the indices of the sensor locations,

S the M×N singular value matrix with min(M,N) real singular values in the diagonal and otherwise vanishing elements and

V a unitary N×N matrix dependent only on the excitation location indices or, respectively, frequency indices and

H the hermetic conjugation of the appertaining matrix.

The singular values are ordered corresponding to their decreasing numerical value, meaning

s₁≧s₂≧ . . . ≧s_{min(M,N)} (3)

If the q-th column vectors of the matrixes U and V are designated by u_{q},v_{q}, then the alternative tonsorial notation (\mathring{x} designates the tensor product)

$$D = {\sum }_{q = 1}^{min ( M , N} {s}_{q} {\underline{u}}_{q} ⊗ {{v}_{\underline{\_}}}_{q}^{H} \qquad ( 4 )$$

clearly shows that the q-th singular value is exclusively linked with the q-th column vectors of U and V. The single and the double underline in u and v should indicate that it concerns an M- or, respectively, N-dimensional vector.

The M indices of the column vectors u_{q} correspond to the successively numbered indices of the measurement sensors. As a result, these column vectors—as noted above—can be reformed In matrices corresponding to the arrangement of the measurement sensors and represented as two-dimensional measurement value distributions. These column vectors are excitation-independent or frequency-independent orthonormalized basis vectors in M-dimensional data space and are here designated as basis maps or eigenmaps.

For singular value analysis, the number Q_{dom} of the significant singular values is determined that specifies the number of the acting fluorescence sources linearly independent with regard to the excitation type.

A punctiform inhomogeneity in the otherwise homogenous optical medium generates, for example, a singular value spectrum with a significant singular value (Q_{dom}=1).

The associated column vectors u_{q} are considered as basis vectors of a—frequency-independent—Q_{dom}-dimensional signal space in M-dimensional data space. The remaining M-Q_{dom} column vectors are then the basis vector of the orthogonal signal space.

The identification of fluorochrome-marked lesions, i.e., the localization, corresponds to the search for locations or focal point locations of excited signal sources. This search by means of computers requires the subdividing (rastering) of the adopted model medium, which should mathematically reproduce the body region to be examined.

One search strategy is to generate, at each raster location, excitation-independent and frequency-independent model data and/or a model data space with the excitation-independent and frequency-independent lead fields, and to compare this and/or these with the excitation-independent and frequency-independent signal space acquired from the measurement data. Comparison measures can be defined such that they display the degree of the “agreement” between signal space and model data/model data space. Locations at which the measure reaches a local maximum are viewed as locations of actual signal sources.

An alternative second search strategy exists in the comparison between the orthogonal signal domain—also called noise domain in the older literature—and the model data or the model data domain. Comparison measures can then be defined such that they display the degree of the “non-agreement” between the orthogonal signal space and model data/model data domain. Locations at which the measure reaches a local minimum are considered to be locations of actual signal sources.

The model data are given by the lead fields: they are either used directly or post-processed.

An individual lead field represents a model data set that reflects a specific property of the signal source. For example, the lead field of a punctiform fluorescence source describes the measurable light intensity given isotropic light emission by this source.

The entirety of the considered lead fields (number: K) defines, due to its linear independence, a K-dimensional model data space. In other words, the lead fields are non-orthogonal basis vectors of this model data space. Orthogonal basis vectors can be acquired by suitable orthogonalization methods, i.e. by post-processing of the lead fields. They do not change the model data domain. However, new individual model data sets result with the new basis vectors (see above). These basis vectors can be additionally normalized. This ensures that lead fields with different separation behavior can be accounted for in the same manner for localization. In addition, it has the advantage of considering physically dimensionless quantities.

An advantageous lead field post-processing is, for example, to normalize the K lead fields Lₖ(k=1, . . . , K) from equation (1) (processing step 27). The individual guide fields are respectively referenced to their normalization, such that the normalized guide fields Lₖ⁽ⁿ⁾ result as follows:

$${\underline{L}}_{k}^{( n )} = \frac{{\underline{L}}_{k}}{‖ {\underline{L}}_{k} ‖} \qquad ( 4 a )$$

For example, by means of a singular-value decomposition of the M×K lead field matrix L, orthogonalized lead fields are acquired. The normalization is displayed by the index (n).

L⁽ⁿ⁾=(L₁⁽ⁿ⁾, . . . , L_{K}⁽ⁿ⁾)=U_{L}S_{L}V_{L}^{T} (5)

For clarity, the arguments of the lead fields (the spatial vectors of the source location) have been omitted. The first K column vectors U(\overline{r})_{L,k}, (k=1, . . . , K) of the matrix U_{L} are the desired source location-dependent orthonormalized lead fields. In the case of a single lead field, the singular-value decomposition is omitted from equation (5).

For example, for the comparison measure a model data set or the model data domain and the signal or the orthogonal signal domain are known from other biomedical applications, analysis of biometric data, or analysis of electrical trans-admittance data. Such methods are projection methods and angular separation methods.

With the aid of projection matrices, individual model data sets or the model data domain are projected either on the signal space or, respectively, on the orthogonal signal domain, and determined for each raster location of the corresponding projection value.

Based on the algorithm to calculate angles between two sub-spaces, a technique known as the angular method (specified in G. Golub et al., page 584 et seq., the angle between the signal domain or the orthogonal signal domain and individual model data sets or the model data domain are calculated search location by search location. Here, a small angle (thus a small value of the comparison measure) between, for example the signal domain and the model data domain, gives a large “agreement”. A transformation of the comparison measure in the form of a 90° angle then again yields maxima of the comparison function at the location of the actual signal sources. The statements can be correspondingly transferred to angular comparison measures between other sub-spaces.

At each location \overline{r} of the discrete optical model medium, it is tested how large the separation is between the orthogonalized lead field U(\overline{r})_{L,k} and the signal space. A suitable measure is the function

Fₖ(\overline{r})=[Σᵢ₌₁^{Q}^{_{dom}}cᵢu−U_{L,k}]². (6)

The output equation of (6) is the equation to be considered in the sense of the quadratic mean

Σᵢ₌₁^{Q}^{_{dom}}cᵢuᵢ=U_{L,k} k=1, . . . , K. (7)

If the solution for the coefficients cᵢ is used in the evaluation measure, then

Fₖ(\overline{r})=1−[Σᵢ₌₁^{Q}^{_{dom}}(uᵢ^{H},U(\overline{r})_{L,k})]². (8)

This measure corresponds to a projection of the considered lead field on the orthogonal signal domain. Using the projection matrix

P_{OS}=1−Σuᵢ\mathring{x}uᵢ^{H} (9)

projected on the orthogonal signal domain results in

Fₖ(\overline{r})=|P_{OS}U(\overline{r})_{L,k}|². (10)

The actual localization function F is the minimal value of the separations Fₖ. It is defined by

$$F ( \vec{r} ) = {min}_{k} \{ {F}_{k} ( \overline{r} ) \} \qquad ( 11 )$$

The local minima of the localization function are monotonically ordered in ascending order corresponding to their number values. The locations, which are to be associated with the first Q_{dom} local minima, are considered as locations of signal generators.

In the case of a number (M_{sys}) of measurement surfaces, the above-cited calculation steps for the data of each measurement surface are executed separately. An objective function then results per measurement surface according to equation (11). From these individual objective functions, an overall objective function F^{(overall)} can be defined according to

F^{(overall)}(\overline{r})=Σ_{μ=1}^{M}^{_{sys}}F^{(μ)}(\overline{r}) (12)

wherein F^{(μ)} is the objective function of the μ-th measurement surface.

The local minima of the overall localization function are monotonically ordered as above, in ascending order corresponding to their number values. The locations, which are to be associated with the first Q_{dom} local minima, are considered as locations of signal sources. The exemplary embodiment confirms the expectation that, given a plurality of non-trivial arranged measurement surfaces, the local minima of the individual objective functions are clearly formed, and thus make the localization result most reliable.

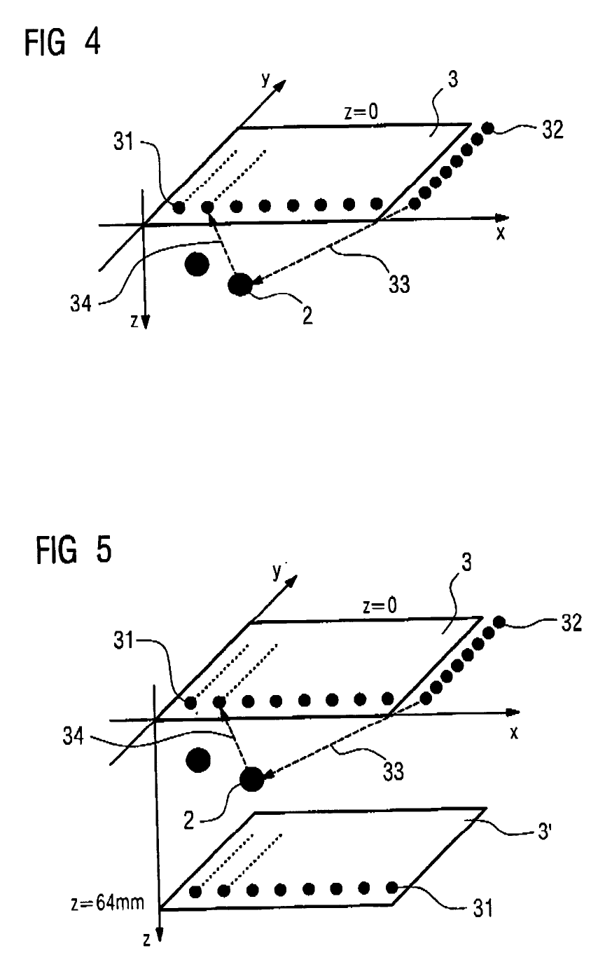

The exemplary embodiments were acquired with planar measurement systems arranged in the applicator 3, which has 8×8 regularly arranged photo sensors 31 as schematically shown in FIG. 4. The sensors 31 were assumed to be punctiform. Their separation along a direction is 8 mm, such that a measurement field surface of 56×56 mm² results. The locations at which 8 laser diodes 32 which radiate the NIR light exciting fluorescence in the body region are located can, for example, be arranged near the measurement surface. The excitation can be, but does not have to be frequency-modulated. Such a measurement arrangement can be guided by hand over a tissue section 1 of interest. The laser diodes 32 emit excitation rays 33 that impinge upon the fluorescing spatial area 2. The fluorescence rays 34 are acquired by the photo sensors 31.

In FIG. 5, a double system of an applicator 3 is shown, with two planar measurement surfaces of the same dimensions (8×8 sensors) arranged opposite one another. For example, they can be integrated into the pressing plate of an x-ray mammography device. The fluorescence excitation ensues at 8 excitation locations that are located near the measurement surface (z=0) of the upper applicator 3. The separation of the two applicators 3 is 64 mm.

As an optical tissue model, in the present invention the following models are used:

A) The simplest model is a borderless area with punctiform fluorescing subjects, which is otherwise optically homogenous (constant optical parameters such as absorption coefficient and scatter coefficient).

B) As a second model, an optically inhomogeneous cuboid area with punctiform fluorescing subjects was considered. It was assumed that absorption coefficient and scatter coefficient can vary locally by 100%. FIG. 9 shows the localization functions of 32 mm and 48 mm deep, fluorochrome-marked lesions. The absorption contrast difference of the surrounding tissue is 100% (image in image).

The simulation of the data is based on the following configurations:

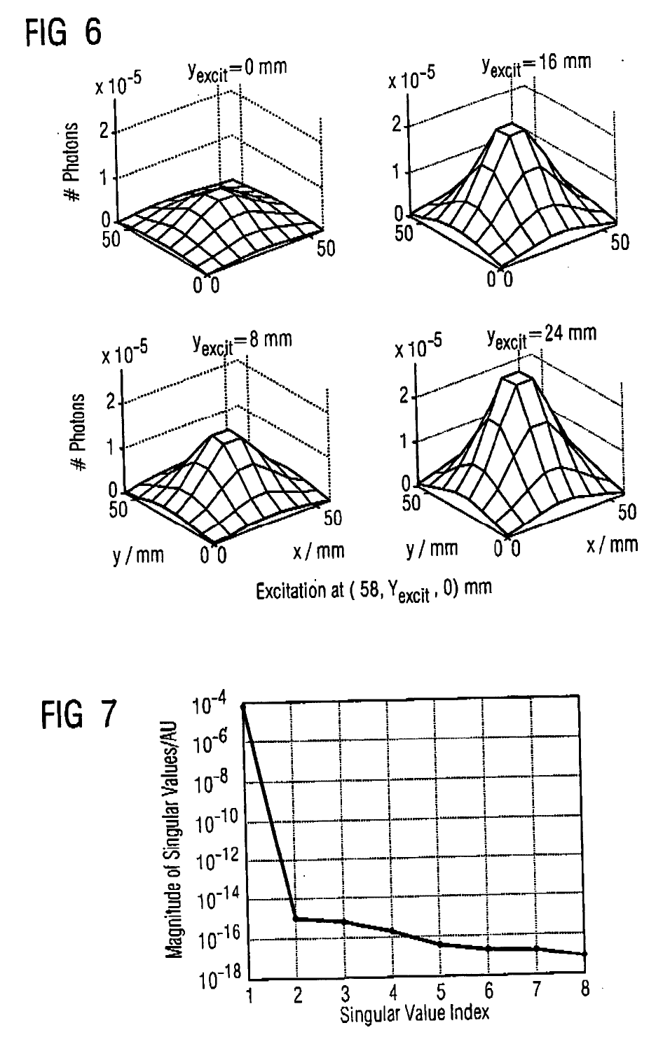

Configuration 1

| | |
|---|---|
| Measurement/excitation system: | see FIG. 4, the excitation is not |
| | frequency-modulated |
| Tissue model: | inhomogeneous cuboid (5.2.B) |
| Fluorescence source: | location at (x, y, z) = (28, 28, 32) mm, |
| | meaning central position beneath the |
| | measurement surface at a depth of |
| | 32 mm (coordinate system see |
| | FIG. 4) |
| Data: | see FIG. 6 |

Configuration 2

| | |
|---|---|
| Measurement/excitation system: | see FIG. 4, the excitation is not |
| | frequency-modulated |
| Tissue model: | inhomogeneous cuboid (5.2.B) |
| Fluorescence source: | location at (x, y, z) = (28, 28, 48) mm, |
| | meaning central position beneath the |
| | measurement surface at a depth of |
| | 48 mm (coordinate system see |
| | FIG. 4) |

Configuration 3a, 4a and 5a

| | |
|---|---|
| Measurement/excitation system: | individual measurement system, see |
| | FIG. 4, the excitation is not |
| | frequency-modulated |
| Tissue model: | homogenous, unbordered medium |
| | (5.2.A) |
| Individual fluorescence sources: | locations at (x, y, z) = (28, 28, 16) mm, |
| | (28, 28, 32) mm, (28, 28, 48) mm, |
| | meaning central positions beneath |
| | the measurement surface at depths |
| | of 16 mm, 32 mm and 48 mm |
| | (coordinate system see FIG. 4) |

Configuration 3b, 4b and 5b

| | |
|---|---|
| Measurement/excitation system: | double measurement system, see |
| | FIG. 5, the excitation is not |
| | frequency-modulated |
| Tissue model: | homogenous, unbordered medium |
| | (5.2.A) |
| Individual fluorescence sources: | locations at (x, y, z) = (28, 28, 16) mm, |
| | (28, 28, 32) mm, (28, 28, 48) mm, |
| | meaning central positions beneath |
| | the measurement surface at depths |
| | of 16 mm, 32 mm and 48 mm |
| | (coordinate system see FIG. 5) |

Due to the singular-value decomposition 23 (corresponding to the number of the existing fluorescence sources), the singular value spectrum shown in FIG. 7 of the data of the configuration 1 comprises a numerically dominant singular value. The remaining singular values reproduce noise, in this case numeric noise.

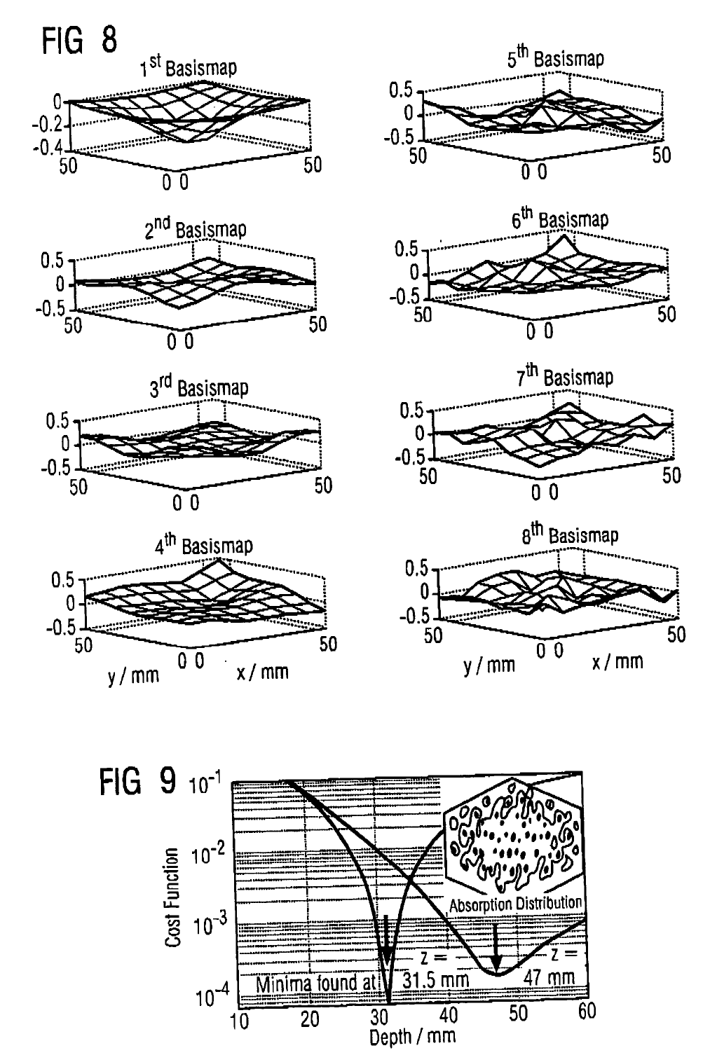

FIG. 8 shows the associated basis maps or eigenmaps. There is a structured basis map corresponding to the single numerically dominant singular value. Here it defines the one-dimensional signal domain of the (here 64-dimensional) data space.

The above-defined objective functions for localization 25, meaning the localization functions, the configurations 1 and 2, are shown in FIG. 9.

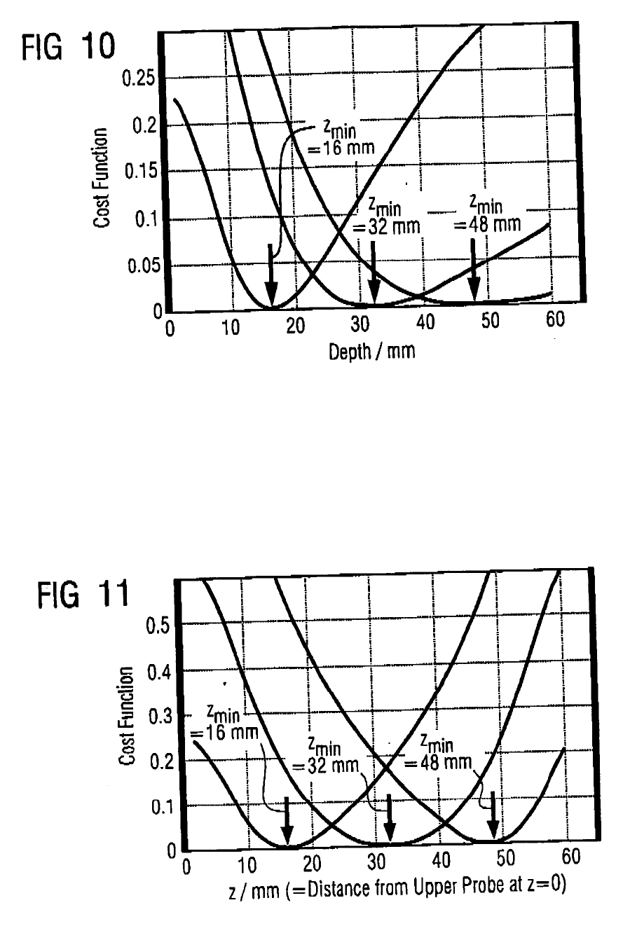

The influence of a second, oppositely-placed measurement surface on the localization is shown using the objective functions of the configurations 3a/b, 4a/b, 5a/b, whereby FIG. 10 shows the localization of lesions of different depths with a planar measurement system, and FIG. 11 shows the localization of lesions of different depths with two planar measurement systems lying opposite one another. The positions of the measurement probes are marked by thick lines at the left edge or, respectively, at both sideways edges. With reference to FIG. 10, a clearer specification of the minima is visible. It should be noted that the scale according to FIG. 11 is different from that of FIG. 10.

The problem of the localization of fluorescing subjects in optically bleary media can be rapidly solved with the inventive method. Furthermore, the precision is increased by the variation of the excitation location.

This inventive localization method operates in real time, is patient-independent, and is robust with regard to estimation of optical parameters.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to localize regions in a biological tissue section that, at least during an examination, exhibits a fluorescence property different from the tissue section, due to which, given an exposure with light of a first wavelength, light of another wavelength is emitted, comprising the steps of:
(a) applying a sequence of fluorescence-exciting light signals at different locations on the tissue-section;
(b) measuring fluorescence light arising due to the light signals, at a plurality of measurement locations on a surface of the tissue section, and thereby obtaining response signals;
(c) determining frequency-independent signal portions in the response signals and further processing the frequency-independent signal portions into input values for localization;
(d) modeling the tissue section and determining a set of guide fields from the model; and
(e) transforming the guide fields and comparing the input values processed from the frequency-independent signal portions with the transformed guide fields, and emitting a location of the transformed guide fields that best reproduces the frequency-independent signal portions as a location of the region to be localized.

2. A method as claimed in claim 1, comprising marking the regions with fluorescing markers to generate the various fluorescence properties.

3. A method as claimed in claim 1 wherein step (a) comprises generating the fluorescence-exciting light signals with various modulation frequencies and radiating the light signals into the tissue section.

4. A method as claimed in claim 3 comprising radiating the fluorescence-exciting light signals as laser light of suitable wavelength.

5. A method as claimed in claim 1, comprising normalizing said guide fields before step (e).

6. A method as claimed in claim 1, wherein step (e) comprises transforming the guide fields into orthogonal guide fields.

7. A method as claimed in claim 6, comprising determining the orthogonal guide fields from the guide fields by a singular-value decomposition.

8. A method as claimed in claim 7, comprising determining optical parameters with reference measurements in non-fluorescence-exciting wavelengths by estimation.

9. A device for localizing regions in a biological tissue section, said biological tissue section, at least during an examination, exhibiting a fluorescence property different from the tissue section, said device comprising:
an arrangement of light sensors distributed on a surface of the tissue section;
a laser diode arrangement for emitting fluorescence-exciting light that interacts with a fluorescing marked region in the tissue section, causing the marked region to emit fluorescence-exited light that is detected by the light sensors in a two-dimensional measurement value distribution, said light sensors generating response signals corresponding to said two-dimensional measurement value distribution; and
a processor supplied with said response signals, said processor determining frequency-independent signal portions in the response signals and further processing the frequency-independent signal portions into input values for localization, modeling the tissue section and determining a set of guide fields from the model; and transforming the guide fields and comparing the input values processed from the frequency-independent signal portions with the transformed guide fields, and emitting a location of the transformed guide fields that best reproduces the frequency-independent signal portions as a location of the region to be localized.

11. A device as claimed in claim 9 wherein said arrangement of light sensors comprises a first set of light sensors and a second set of light sensors adapted to be respectively disposed on opposite sides of said tissue section.

12. A device as claimed in claim 9 comprising an x-ray mammography apparatus having two compression plates, and wherein said light sensor arrangement is integrated into at least one of said compression plates.

13. A device as claimed in claim 1 wherein said arrangement of light sensors comprises a flexible mounting for said light sensors.

14. A device as claimed in claim 9 wherein said arrangement of light sensors comprises a curved mounting for said light sensors.
